(19) United States
(12) Patent Application Publication
(10) Pub. No.: US 2015/0327799 A1
(43) Pub. Date: Nov. 19, 2015

(54) SYSTEMS AND METHODS FOR MEASUREMENT OF OXYGEN LEVELS IN BLOOD BY PLACEMENT OF A SINGLE SENSOR ON THE SKIN

(71) Applicant: NuLine Sensors, LLC, Lake Mary, FL (US)

(72) Inventors: Michael J. Vosch, Titusville, FL (US); Jonathan R. Mohlenhoff, Longwood, FL (US); Adriana Hernandez, Lake Mary, FL (US); Benjamin M. Downey, Sanford, FL (US)

(21) Appl. No.: 14/714,210

(22) Filed: May 15, 2015

Related U.S. Application Data
(60) Provisional application No. 61/993,301, filed on May 15, 2014.

(51) Int. Cl. A61B 5/1455; A61B 5/00

(52) U.S. Cl. CPC A61B 5/14551; A61B 5/0022; A61B 5/7435

(57) ABSTRACT

Disclosed herein are systems and methods for measurement of oxygen levels in blood by placement of a single sensor on the skin. According to an aspect, a method includes using a light transmitter to generate and direct light into a surface of skin. The method also includes using a detector to receive non-absorbed light. Further, the method includes measuring oxygen saturation level in blood based on the non-absorbed light.

### CROSS REFERENCE TO RELATED APPLICATION

This application claims priority to U.S. Provisional Patent Application No. 61/993,301, filed May 15, 2014 and titled SYSTEMS AND METHODS FOR MEASUREMENT OF OXYGEN LEVELS IN THE BLOOD BY THE PLACEMENT OF A SINGLE SENSOR ON THE SKIN; the disclosure of which is incorporated herein by reference in its entirety.

### TECHNICAL FIELD

The present disclosure relates to systems and techniques for sensing oxygen levels in the blood. More particularly, the present disclosure relates to systems and methods for measurement of oxygen levels in blood by placement of a single sensor on the skin.

### BACKGROUND

Pulse oximetry is a non-invasive method for monitoring a patient's oxygen saturation. Most commonly, a sensor is placed on a thin part of the patient's body, usually a fingertip or earlobe, or in the case of an infant, across a foot. Light of two wavelengths is then passed through the patient to a photodetector. The changing absorbance at each of the wavelengths is measured, allowing determination of the absorbances due to the pulsing arterial blood alone, excluding venous blood, skin, bone, muscle, and fat. Pulse oximetry is a particularly convenient non-invasive measurement method.

A pulse oximeter is a medical device that indirectly monitors the oxygen saturation of a patient's blood (as opposed to measuring oxygen saturation directly through a blood sample) and changes in blood volume in the skin, producing a photoplethysmogram. Typically, it utilizes a processor and a pair of small light-emitting diodes (LEDs) facing a photodiode through a translucent part of the patient's body, usually a fingertip or an earlobe. Oxygenated blood or hemoglobin absorbs more infrared light and allows more red light to pass through. Deoxygenated hemoglobin allows more infrared light to pass through and absorbs more red light. A photodiode measures the amount of light that is transmitted and not absorbed. The measurement fluctuates in time because the amount of arterial blood that is present increases with each heartbeat. A processor is typically used to calculate the oxygen levels based on the light received at a sensor positioned opposite of the LED provided as a source of the emitted light.

However, all current methods relate to passing light through the skin (e.g., fingers, toes, sublingual pocket, the middle flap of tissue in nose, etc.) where the light is transmitted from one side of the portion of the body and received at a second or opposite side or surface of the portion of the body. These methods calculate oxygen saturation based on what light is transmitted from one side of the selected skin site through to the detector on the other side of the skin site (e.g., transcutaneous). In this manner, it is necessary to place a sensor opposite the source for the light. Thus, a portion of the body thin enough to allow the transmission or passing of the light is necessary. If it is desired to measure oxygen saturation on a portion of the body that is not thin enough to allow the transmission of light (e.g., the head, arm, leg, etc.), these methods will provide erroneous results.

In view of the foregoing, there is a need for an improved measurement of oxygen levels in the blood.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are systems and methods for measurement of oxygen levels in blood by placement of a single sensor on the skin. According to an aspect, a method includes using a light transmitter to generate and direct light into a surface of skin. The method also includes using a detector to receive non-absorbed light. Further, the method includes measuring oxygen saturation level in blood based on the non-absorbed light.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. In the drawings:

FIG. 1 is a diagram of an example medical device for measurement of oxygen levels in blood according to embodiments of the present disclosure;

FIG. 2 is a flowchart of an example method for measurement of oxygen levels in blood according to embodiments of the present disclosure;

FIG. 3 is a block diagram of another example medical device according to embodiments of the present disclosure;

FIG. 4 is a graph depicting the absorption of oxygenated and deoxygenated hemoglobin at different light wavelengths;

FIG. 5 is a graph showing an oxygen level in blood curve in accordance with embodiments of the present disclosure; and

FIG. 6 is a diagram of another example medical device in accordance with embodiments of the present disclosure.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

As referred to herein, the term “computing device” should be broadly construed. It can include any type of device including hardware, software, firmware, the like, and combinations thereof. A computing device may include one or more processors and memory or other suitable non-transitory, computer readable storage medium having computer readable program code for implementing methods in accordance with embodiments of the present disclosure. In another example, a computing device may be a server or other computer and communicatively connected to other computing devices (e.g., handheld devices or computers) for data analysis. In another example, a computing device may be a mobile computing device such as, for example, but not limited to, a smart phone, a cell phone, a pager, a personal digital assistant (PDA), a mobile computer with a smart phone client, or the like. In another example, a computing device may be any type of wearable computer, such as a computer with a head-mounted display (HMD). A computing device can also include any type of conventional computer, for example, a laptop computer or a tablet computer. A typical mobile computing device is a wireless data access-enabled device (e.g., an iPHONE® smart phone, a BLACKBERRY® smart phone, a NEXUS ONE™ smart phone, an iPAD® device, or the like) that is capable of sending and receiving data in a wireless manner using protocols like the Internet Protocol, or IP, and the wireless application protocol, or WAP. This allows users to access information via wireless devices, such as smart phones, mobile phones, pagers, two-way radios, communicators, and the like. Wireless data access is supported by many wireless networks, including, but not limited to, CDPD, CDMA, GSM, PDC, PHS, TDMA, FLEX, ReFLEX, iDEN, TETRA, DECT, DataTAC, Mobitex, EDGE and other 2G, 3G, 4G and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone that operates over GPRS (General Packet Radio Services), which is a data technology for GSM networks. In addition to a conventional voice communication, a given mobile device can communicate with another such device via many different types of message transfer techniques, including SMS (short message service), enhanced SMS (EMS), multi-media message (MMS), email WAP, paging, or other known or later-developed wireless data formats. Although many of the examples provided herein are implemented on smart phone, the examples may similarly be implemented on any suitable computing device, such as a computer.

As referred to herein, the term “user interface” is generally a system by which users interact with a computing device. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

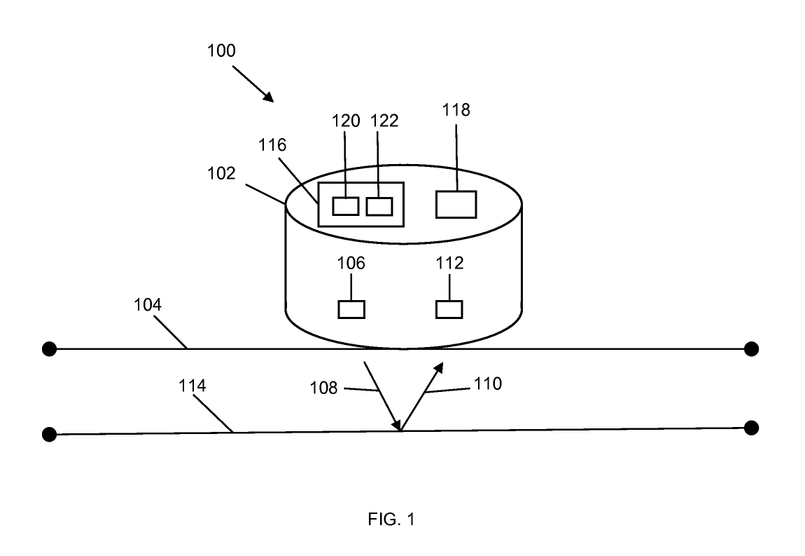

FIG. 1 illustrates a diagram of an example medical device 100 for measurement of oxygen levels in blood according to embodiments of the present disclosure. Referring to FIG. 1, the medical device 100 includes a sensor 102 placed on a surface of skin 104 of a person according to embodiments of the present disclosure. The sensor 102 may alternatively be placed near the surface of the skin 104. The sensor 102 includes a light transmitter 106 configured to transmit light generally in a direction indicated by arrow 108. More particularly, the light transmitter 106 may generate light of multiple wavelengths. In an example, the generated light may be in the red and/or infrared light spectrums. As an example, red light has a wavelength of approximately 650 nanometer (nm). However, it is noted that other light with wavelengths of 200 nm to 900 nm may also or alternatively be used. As a further example, infrared light, having wavelengths of 700 nm to 1.8 millimeter (mm) may be also or alternatively be used. The light of multiple wavelengths may be simultaneously transmitted. The light may be directed to and transmitted through the skin 104 and into the person's body. All or a portion of the light may be reflected in a direction generally indicated by arrow 110. LED emitters of various wavelengths of light may be used to allow for the proper measurement of the of the oxygen levels. According to embodiments, a light transmitter may include one or more LEDs in the range of red light, and one or more LEDs in the range of infrared light.

The sensor 102 may include a detector 112 positioned and configured to receive the reflected light. The light generated by the light transmitter 106 may be directed at an angle such that the light is reflected from within the body of the person and received by the detector 112. Because of physical properties of infrared light and ability to travel through skin, it may be desirable to place the sensor 102 on an area of the skin 104 where there is minimal tissue between the outer layer of the skin 104 and bone 114 of the person. One non-limiting example is placement of the sensor 102 on the forehead of a person. The sensor 102 may be configured to measure and analyze the reflected light to determine oxygen saturation levels in the blood of the person. Example detectors include, but are not limited to, photodetectors, a pin diode, a photo diode, a CCD, or other type of detector may be used; however, the operational range of the detector may be consistent with the wavelengths of the light transmitters used.

With continued reference to FIG. 1, the sensor 102 may include a computing device 116 and an internal power source 118. The computing device 116 and the power source 118 may be operatively connected to the light transmitter 106 and the detector 112. The power source 116 may provide power to the computing device 114, the light transmitter 106 and the detector 112. The computing device 114 may include hardware, software, firmware, or combinations thereof for implementing the functions disclosed herein. For example, the computing device 114 may include a processor 120 and memory 122. In an example, the computing device 116 may also be positioned external to the sensor 102. The computing device 116 may be configured to control the output of the light from the light transmitter 106. The computing device 116 may also be configured to receive signals from the detector 112 for analyzing the oxygen saturation levels and other metrics of the blood.

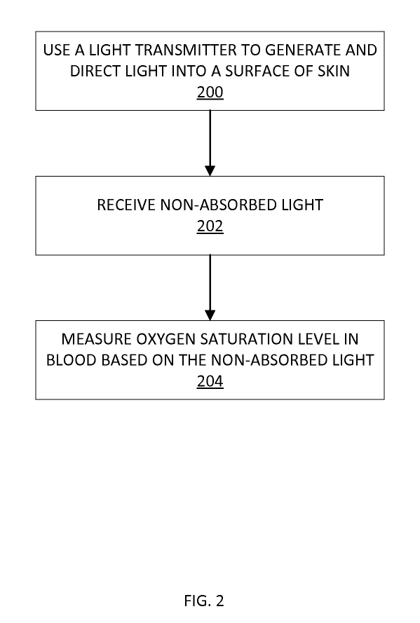

FIG. 2 illustrates a flowchart of an example method 200 for measurement of oxygen levels in blood according to embodiments of the present disclosure. The method is described in this example as being implemented by the medical device 100 shown in FIG. 1, although it should be understood that the method may be implemented by any other suitable device. Also, it is noted that the computing device 116 may be suitably configured to control the light transmitter 106 and the detector 118 for implementing the functionality of the method.

Referring to FIG. 2, the method includes using 200 a light transmitter to generate and direct light into a surface of skin. For example, the computing device 116 shown in FIG. 1 may be configured to control the light transmitter 106 to generate and transmit light into the skin 104. The light may generally be directed to transmit in the direction of arrow 108. The light may penetrate through tissue until it reaches bone 114 where it is reflected towards the detector 102. The light may also be reflected by tissue towards the detector 112.

The method of FIG. 2 includes receiving 202 non-absorbed light. Further, the method includes measuring oxygen saturation level in blood based on the non-absorbed light. Continuing the aforementioned example, the detector 112 may receive light that is not absorbed by the tissue or bone 114 of the person. The detector 112 may generate a signal representative of the received light. The computing device 116 may be communicatively connected to the detector 112 for receipt of the generated signal. Further, the computing device 116 may measure an oxygen saturation level of the blood. The sensor 102 may operate to continuously measure the oxygen saturation level over time. Alternatively, the oxygen saturation level may be measured at different times (e.g., periodically).

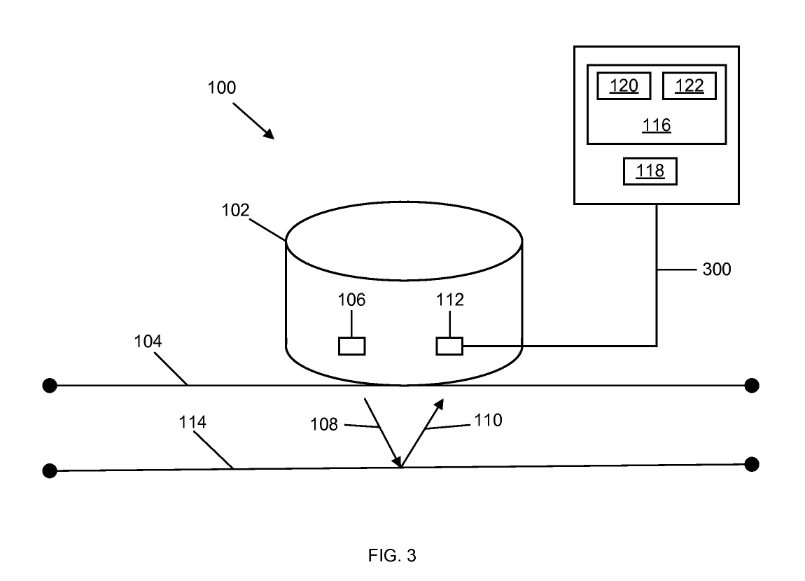

FIG. 3 illustrates a block diagram of another example medical device 100 according to embodiments of the present disclosure. The medical device 100 shown in FIG. 3 is similar to the medical device 100 shown in FIG. 1 except that the computing device 116 and the power source 118 are located outside of the sensor 102 packaging. The computing device 116 and the power source 118 may be operatively connected to the light transmitter 106 and the detector 112 via suitable cabling 300. Alternative to cabling 300, the computing device 116 may be operatively connected to the light transmitter 106 and the detector 112 via a suitable wireless connection, such as by use of BLUETOOTH® or WIFI® communications technology.

In experimentations, a device as disclosed herein has a peripheral capillary oxygen saturation (SpO2) measuring range of 35%-99%. Further, the device has been shown to have an accuracy of (+/−)2% or (+/−)2 bpm (beats per minute) (during 75%-99%).

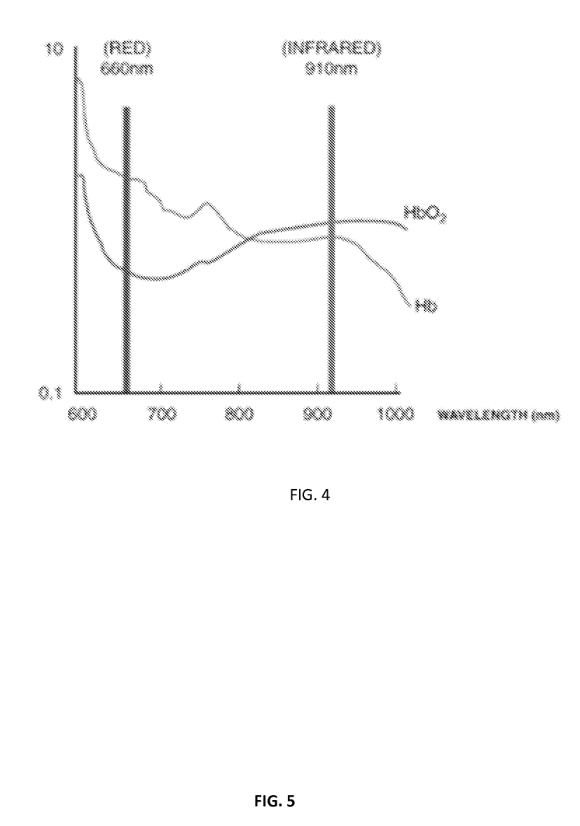

It is noted that the principle of pulse oximetry is based on the red and infrared light absorption characteristics of oxygenated and deoxygenated hemoglobin. Oxygenated hemoglobin absorbs more infrared light and allows more red light to pass through. Deoxygenated (or reduced) hemoglobin absorbs more red light and allows more infrared light to pass through. Red light is in the 600-700 nanometer (nm) wavelength light band. Infrared light is in the 850-1000 nm wavelength light band. FIG. 4 illustrates a graph depicting the absorption of oxygenated and deoxygenated hemoglobin at different light wavelengths.

In consideration of development of the devices and methods disclosed herein, it is noted that arterial blood is of interest, but at the measuring site there are constant light absorbers that are present. Example light absorbers include, but are not limited to, skin, tissue, venous blood, and arterial blood. However, with each heartbeat, the heart contracts and there is a surge of arterial blood, which momentarily increases arterial blood volume across the measuring site. This can result in more light absorption during the surge. If light signals received at the detector are viewed as a waveform, there is expected to be peaks with each heartbeat and troughs between heartbeats. If the light absorption at the trough (which include all the constant absorbers) is subtracted from the light absorption at the peak, then the resultants are the absorption characteristics due to added volume of blood only, which is arterial.

SaO₂ is defined as the ratio of the level oxygenated hemoglobin over the total hemoglobin level (oxygenated and depleted):

$${SaO}_{2} = \frac{{HbO}_{2} + Hemoglobin}{Total Hemoglobin}$$

Body tissue absorbs different amounts of light depending on the oxygenated level of blood that is passing through it. This characteristic is nonlinear. The formula above is the normal ratio of oxygenated/deoxygenated hemoglobin that is present at any given moment passing under the detecting device. Once the readings from the sensor are taken the value as a percentage of O2 Saturation is then calculated by the system so the end user sees only the final numbers and does not need to perform the calculations.

The red wavelength may be about 660 nm and the infrared wavelength may be about 880 nm. The ratio of the arterial blood reflection at these wavelengths may be proportional to the amount of oxygen in the blood.

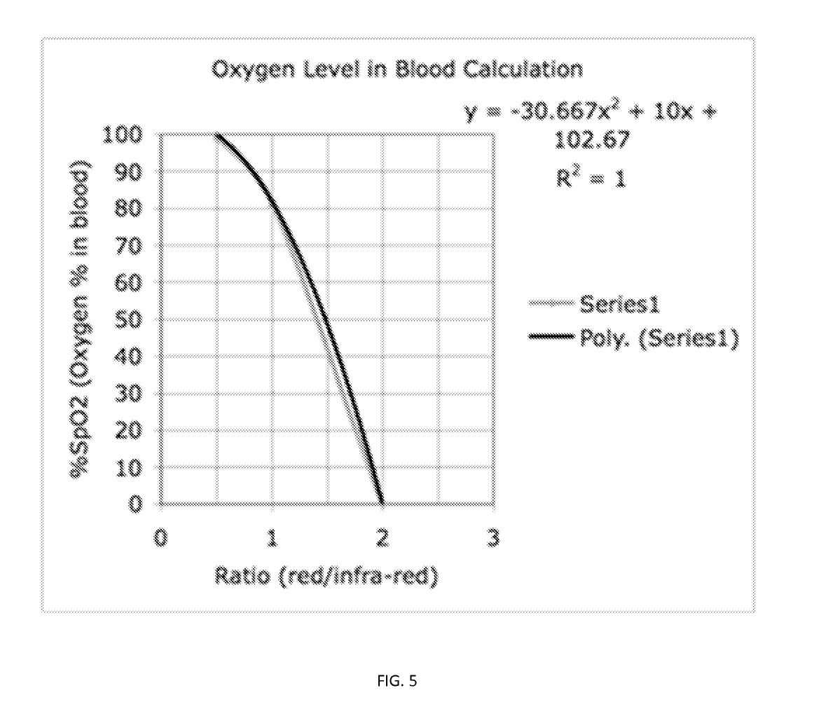

FIG. 5 illustrates a graph showing an oxygen level in blood curve in accordance with embodiments of the present disclosure. The curve shows the oxygenated hemoglobin over time. This curve is used to demonstrate how the human body's circulatory system is moving oxygenated/deoxygenated hemoglobin to show the changes with temperature, pulse pressure and Ph factors. The formula for percentage oxygen in blood may be set forth as follows:

% HbO₂=−30.667*ratio²+10*ratio+102.67

where ratio is the ratio of the reflectance of the arterial blood at 660 nm divided by the reflectance of the arterial blood at 880 nm.

An oximeter is operable to measure the oxygen saturation of hemoglobin in arterial blood. The oximeter may include, for example, a sensor, a microprocessor unit for processing signals from the sensor, and a display.

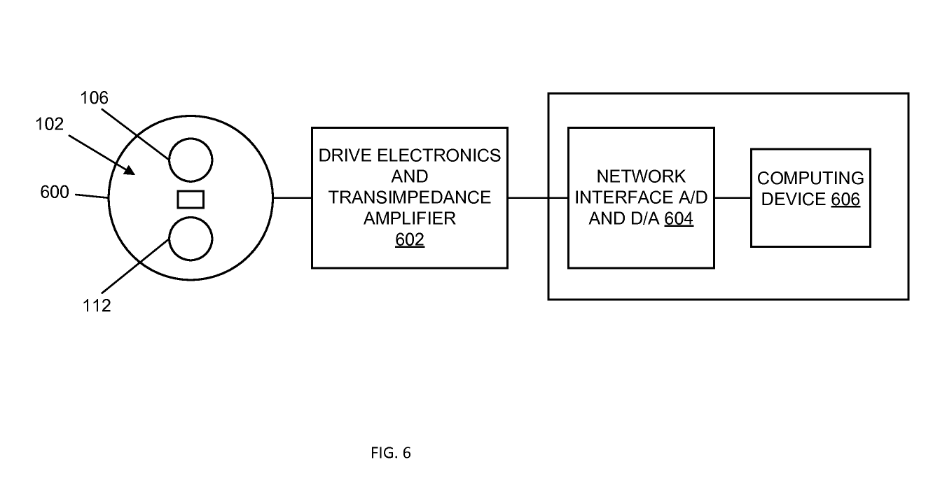

FIG. 6 illustrates a diagram of another example medical device 100 in accordance with embodiments of the present disclosure. Referring to FIG. 6, the medical device 100 includes a patch 600 including a sensor 102 having a light transmitter 106 and a detector 112. The patch 600 may be suitably configured with an adhesive or the like for attaching to a person's chest or other suitable area of the body. The device 100 may include drive electronics and a transimpedance amplifier 602 for suitably connected (e.g., either wired or wireless connection) to the sensor 102 for conditioning a signal output by the detector 112. Further, the medical device 100 includes a network analog-to-digital (A/D) and digital-to-analog (D/A) connector 604 for interfacing with a computing device 606. The computing device may have suitable reader software. The connector 604 may be operatively connected to the computing device 606 via a universal serial bus (USB) interface. A few of the devices that may use the devices and systems disclosed herein are congestive heart failure monitors, sleep apnea monitors, emergency medical monitors, cardiac rescue devices, cardio-pulmonary resuscitation devices and other devices where the end-user would like a non-invasive way of determining the level of oxygenation of blood in the human body.

The formula used has been the standard model for humans and the formula takes into account the various changes in pulse pressures, blood pH, and temperature factors. As shown in the graph in FIG. 5 the ratio is the amount of red emitter reflection divided by the amount of infrared emitter reflection as determined by the detector 112. As the ratio moves from 0 toward 2 the amount of % SpO2 moves from 100% toward 0%.

The various techniques described herein may be implemented with hardware or software or where appropriate, with a combination of both. Thus, the methods and apparatus of the disclosed embodiments, or certain aspects or portions thereof, may take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for practicing the presently disclosed subject matter. In the case of program code execution on programmable computers, the computer can generally include a processor, a storage medium readable by the processor (including volatile and non-volatile memory and/or storage elements), at least one input device and at least one output device. One or more programs may be implemented in a high level procedural or object oriented programming language to communicate with a computer system. However, the program(s) can be implemented in assembly or machine language, if desired. In any case, the language may be a compiled or interpreted language, and combined with hardware implementations.

The described methods and apparatus may also be embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, a video recorder or the like, the machine becomes an apparatus for practicing the presently disclosed subject matter. When implemented on a general-purpose processor, the program code combines with the processor to provide a unique apparatus that operates to perform the processing of the presently disclosed subject matter.

Features from one embodiment or aspect may be combined with features from any other embodiment or aspect in any appropriate combination. For example, any individual or collective features of method aspects or embodiments may be applied to apparatus, system, product, or component aspects of embodiments and vice versa.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. A method comprising:
using a light transmitter to generate and direct light into a surface of skin;
using a detector to receive non-absorbed light; and
measuring oxygen saturation level in blood based on the non-absorbed light.

2. The method of claim 1, wherein the light is one of in the red light spectrum and the infrared light spectrum.

3. The method of claim 1, wherein measuring the oxygen saturation level comprises using at least one of a processor and memory to measure the oxygen saturation level based on the non-absorbed light.

4. The method of claim 1, further comprising transmitting the measured oxygen saturation level.

5. The method of claim 4, wherein transmitting the measured oxygen saturation level comprises wirelessly transmitting the measured oxygen saturation level.

6. The method of claim 4, wherein transmitting the measured oxygen saturation level comprises transmitting the measured oxygen saturation level via a wired connector.

7. The method of claim 1, further comprising power the light transmitter and the detector.

8. A medical device comprising:
a light transmitter configured to generate and direct light into a surface of skin;
a detector configured to receive non-absorbed light; and
at least one processor and memory configured to measure oxygen saturation level in blood based on the non-absorbed light.

9. The medical device of claim 8, wherein the light is one of in the red light spectrum and the infrared light spectrum.

10. The medical device of claim 8, wherein the at least one processor and memory is configured to measure the oxygen saturation level based on the non-absorbed light.

11. The medical device of claim 8, further comprising circuitry configured to transmit the measured oxygen saturation level.

12. The medical device of claim 11, wherein the circuitry is configured to wirelessly transmit the measured oxygen saturation level.

13. The medical device of claim 11, wherein the circuitry comprises a wired connector configured to wirelessly transmit the measured oxygen saturation level.

14. The medical device of claim 8, further comprising a power source configured to power the light transmitter and the detector.
